(19) United States
(12) Patent Application Publication
(10) Pub. No.: US 2022/0039920 A1
(43) Pub. Date: Feb. 10, 2022

(54) PERSONAL WEARABLE LOCATION AND WEAR TIME TRACKING DEVICE

(71) Applicant: IOT MED/DENT SOLUTIONS LLC, Park City, UT (US)

(72) Inventors: Richard Kreifeldt, Park City, UT (US); Robert Miller, Park City, UT (US); Juliana Miller, Park City, UT (US)

(73) Assignee: IOT MED/DENT SOLUTIONS LLC, Park City, UT (US)

(21) Appl. No.: 16/985,554

(22) Filed: Aug. 5, 2020

(51) Int. Cl. A61C 7/08; H04W 4/38; H04W 4/029; G08B 21/24; H04W 4/02; H04W 4/70; H02J 7/02; G01K 3/00; G01K 13/00; G01L 1/22; G01N 27/22; A61B 5/00; A61B 5/0205

(52) U.S. Cl. CPC A61C 7/08; G16Y 10/60; H04W 4/029; G08B 21/24; H04W 4/026; H04W 4/023; H04W 4/70; H02J 7/02; G01K 3/005; G01K 13/002; G01L 1/22; G01N 27/223; A61B 5/6844; A61B 5/02055; A61B 5/6846; A61B 5/0002; H04W 4/38

(57) ABSTRACT

An electronic personal wearable device is disclosed which includes a processor, a transceiver, and a wearing sensor which indicates that the electronic personal wearable device is worn by the user when the electronic personal wearable device is worn by the user. A system includes the electronic personal wearable device and a personal electronic device. The electronic personal wearable device may, in at least one implementation, be an orthodontic retainer.

### BACKGROUND

In relatively modern history, personal wearable devices have been developed to assist people with ailments, injuries, and diseases as well as assisting with scheduling, directions, and personal tasks. For example, as glass became cheaper, the first primitive eyeglasses were developed in the 14^{th} century to correct the sight of people with eye ailments. Immobilizing leg braces were as simple as sturdy sticks lashed to a person's leg to prevent movement of the knee or to provide weight bearing support for the braced leg. Further, some diseases were treated with medications stored in wearable containers to allow the user to always have medication on their person. More recently, pocket watches, digital watches, and now, smart watches, have been typically worn on the wrist or in a pocket to tell the user what time it is, to where they are navigating, and what tasks are to be done on a particular day. Other wearable devices are worn by sick or elderly people which summon help from emergency services in response to a button push.

One of the problems with wearable personal devices has always been becoming separated from the wearable personal devices. For example, eyeglasses left at home do not help a student see a chalk board during classroom instruction. Similarly, watches, hearing aids, personal locator beacons, and other personal wearable devices are subject to being lost, forgotten, or misplaced. Historically, these personal wearable devices were such an expense that a lost pocket watch, for example, would take months of salary to replace. More recently, many of these devices are still costly to simply replace when lost. One solution to this problem, which has been a fairly recent improvement with electronic devices, is position tracking of the device through a smart phone, for example. Hearing aids, for example, may be trackable through a computer or a smart phone when the hearing aids are pinged by the smartphone for location detection.

Unfortunately, location detection is not readily available on many devices for various reasons, such as location detection technology costs more to implement than replacement of the device would cost. Further, this type of location detection can only help a user find hearing aids when they have been lost by the user and then, only for as long as they maintain electrical power sufficient to power the hearing aids.

A further problem of personal wearable devices is that they are frequently removed from a person's body due to discomfort or annoyance or are removed intentionally and not reinstalled because of forgetfulness. As such, a wearer of the personal wearable device may fail to realize the clinical benefit of the device. For example, if an orthodontic retainer is not worn for a sufficient amount of time, the entire orthodontic effort expended on moving teeth in a person's mouth may be negated and the teeth may return to their original orientation. At the time, the wearer of the personal wearable device may fail to appreciate that due to removal of the personal wearable device, the clinical benefit of the device over the long term may be lost.

Accordingly, it is one aspect of this disclosure to provide an electronic personal wearable device which may include circuitry to allow the electronic personal wearable device to be tracked by location. It is a further object of this disclosure to provide an electronic personal wearable device which may include circuitry to allow the electronic personal wearable device to be tracked for wear time.

### SUMMARY OF THE DISCLOSURE

Disclosed below is a device which includes a processor, a transceiver, and a wearing sensor which indicates the device is being worn by a user when the device is worn by the user.

A system is further disclosed which includes an electronic personal wearable device which comprises a processor, a transceiver, and a wearing sensor which indicates the device is being worn by a user when the device is worn by the user. The system may further include a personal electronic device.

Also disclosed herein is a device. The device may be an orthodontic retainer and include a processor, a transceiver, and a wearing sensor which indicates the device is being worn by a user when the device is worn by the user

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. Advantages of the disclosure will become better understood with regard to the following description and accompanying drawings where:

FIG. 1A illustrates a personal wearable device.

FIG. 1B illustrates a sensor included in the personal wearable device.

FIG. 2 illustrates a system for that facilitates communication between the personal wearable device and a personal electronic device or cloud server.

FIG. 3 illustrates a system for communicating with the personal wearable device.

FIG. 4 illustrates a system for storing information generated by the personal wearable device.

FIG. 5 illustrates a method for tracking a location of the personal wearable device.

FIG. 6 illustrates a method for tracking a location of the personal wearable device.

FIG. 7 illustrates a method for tracking wear time of the personal wearable device.

FIG. 8 illustrates a method for identifying that the personal wearable device is being worn.

### DETAILED DESCRIPTION

In the following description of the disclosure, reference is made to the accompanying drawings, which form a part hereof, and in which is shown by way of illustration specific implementations in which the disclosure is may be practiced. It is understood that other implementations may be utilized, and structural changes may be made without departing from the scope of the disclosure.

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure, may be alternatively included in another embodiment or figure regardless of whether or not those elements are shown or described in another embodiment or figure. In other words, elements in the figures may be interchangeable between various embodiments disclosed herein, whether shown or not.

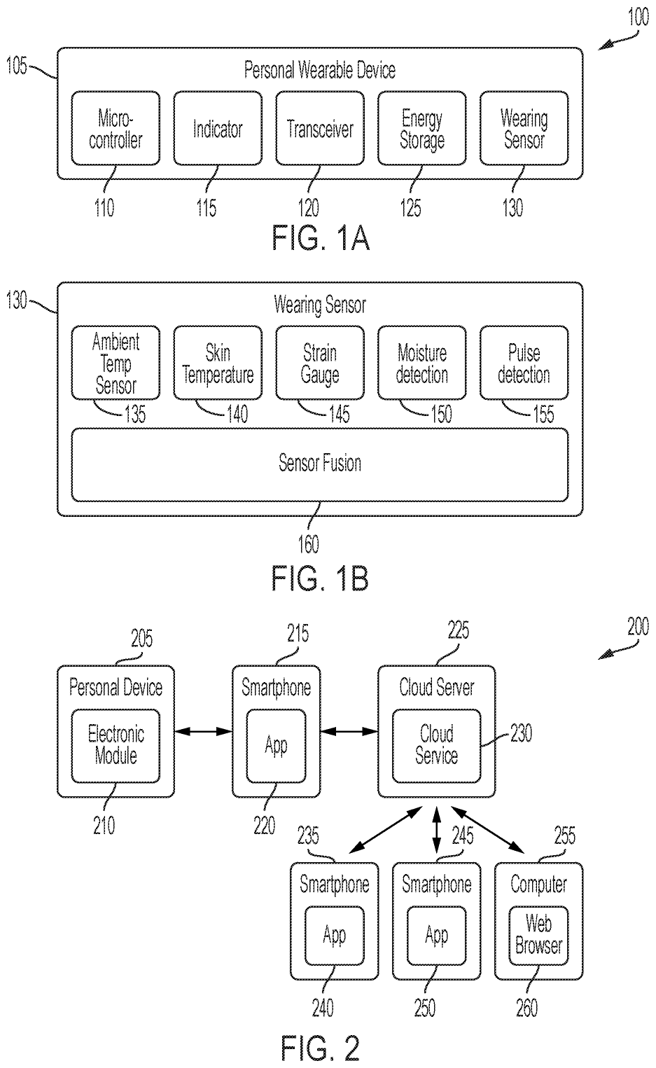

FIG. 1 illustrates a personal wearable device 100 including an electronic personal wearable device 105. An electronic personal wearable device may be used herein to identify any electronic wearable device. Examples of wearable personal devices may include electronic and non-electronic wearable devices. Examples of an electronic personal wearable device may include retainers for teeth, removable bridges, dentures, eyeglasses and hearing aids which are fitted with electronic devices described herein. Other non-electronic wearable devices may include an ankle brace or knee brace or any other wearable device, and may be integrated with electronics suitable to perform the function and features of electronic personal wearable device 105 disclosed herein.

Electronic personal wearable device 105 may include a microcontroller 110, an indicator 115, a transceiver 120, an energy storage 125, and a wearing sensor 130. Microcontroller 110 may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. Microcontroller 110 may execute pre-programmed instructions to perform functionalities described below.

Indicator 115 may be implemented simply as a light (e.g., a light emitting diode) or other display, or may be implemented as an audible indicator such as a piezoelectric speaker. Indicator 115 may operate by interfacing with a personal electronic device, such as a smart phone, tablet, laptop, or desktop computer, and may automatically connect with the personal electronic device to automatically communicate with the device and turn on the indicator which may allow the user to locate electronic personal wearable device 105, as will be discussed below.

Electronic personal wearable device 105 may further include a transceiver 120 which may operate to receive instructions and transmit information from electronic personal wearable device 105. Transceiver 120 may include a number of hardware components such as transmitters, receivers, and antennas. Transceiver 120 may operate using any known network interface communication protocol, including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between electronic personal wearable device 105 and, for example, a smart phone or cloud based service. Transceiver 120 may or may not be implemented within microcontroller 110 and may or may not be implemented on a single silicon chip.

Energy storage 125 may provide electrical power to other components within electronic personal wearable device 105, such as microcontroller 110, indicator 115, and transceiver 120. Energy storage 125 may be implemented as a battery, a super-capacitor, or any other electricity storage method. In one embodiment, a super-capacitor may be implemented within electronic personal wearable device 105 using various layers that make up the body of the specific personal wearable device. For example, an electronic personal wearable device 105 may be a retainer, which is worn in the mouth and is used to retain teeth in a particular location in the mouth, especially after orthodontic treatment. Thus, in the case of a retainer, a super capacitor may be built into the retainer by using, in order, at least a biocompatible plastic layer, an electrically conductive layer, an insulating plastic layer, an electrically conductive layer, and a biocompatible plastic layer. Such an organization of various layers of electronic personal wearable device 105 may be similarly implemented in eyeglasses, removable bridges, dentures, hearing aids, or any other wearable personal electronic device.

Wearing sensor 130 is further discussed with respect to FIG. 1B, below. However, wearing sensor 130 refers to a sensor network which is disposed within electronic personal wearable device 105 (e.g., molded into a plastic of an orthodontic retainer or mouthguard or within the porcelain of a dental bridge, etc.). Wearing sensor 130 includes a plurality of sensors for detecting various conditions for electronic personal wearable device 105 which may provide information about the physical status (e.g., degree of deformity, wear time, and other information. As shown in FIG. 1B, wearing sensor 130 may include one or more of an ambient temperature sensor 135, a skin temperature sensor 140, a strain gauge 145, a moisture detection sensor 150, and a pulse detection circuit 155. Each of one or more of ambient temperature sensor 135, skin temperature sensor 140, strain gauge 145, moisture detection sensor 150, and pulse detection circuit 155 in wearing sensor 130 may be connected to a sensor fusion network 160 which takes information from one or more of ambient temperature sensor 135, skin temperature sensor 140, strain gauge 145, moisture detection sensor 150, and pulse detection circuit 155 and provides the information to microcontroller 110, shown in FIG. 1A.

Ambient temperature sensor 135 may detect a temperature of an ambient environment. For different electronic personal device 105 implementations, the ambient environment may be a room temperature, a temperature inside a person's mouth, a temperature outside, etc. Ambient temperature sensor 135 may detect that the ambient temperature is approximately 98.6 degrees (Fahrenheit) and inside a person's mouth, for example. Skin temperature sensor 140 may be used similarly to detect a temperature of a person's skin, which may be useful in the same or different electronic personal device 105 implementations. Temperature data from either ambient temperature sensor 135 or skin temperature sensor 140 may provide data that is indicative of electronic personal device 105 being worn by a person.

Strain gauge 145 may be a sensor element within wearing sensor 130. Strain gauge 145 may assess strain on a device, for example, by determining an “at rest” strain in free space and a strain level at a time when the device is worn. For example, microcontroller 110 may receive data through sensor fusion network 160 that reflects a change in the “at rest” strain to increased strain and, based on that information, detect that electronic personal wearable device 105 is being worn by a user. Alternatively, sensor fusion network 160 may compare the “at rest” strain to increased strain, and output a signal to microcontroller 110 that indicates that electronic personal wearable device 105 is currently being worn. Strain gauge 145 may also obtain a measurement of strain on electronic personal wearable device 105 and provide that measurement to microcontroller 110 to assess relative deformity of electronic personal wearable device 105.

Moisture detection sensor 150 may be another sensor element within wearing sensor 130 of electronic personal device 105. Moisture detection sensor 150 may detect the presence of humidity or liquid in the environment around moisture detection sensor 150. For example, if electronic personal wearable device 105 is implemented as an orthodontic retainer, moisture detection sensor 150 may detect whether or not electronic personal wearable device 105 is disposed within the mouth of a user at a particular time. Moisture detection sensor 150 may operate on a contact-based system with a resistive element. For example, when the resistive element absorbs moisture, a resistance value of the sensor may be reduced. When the resistance value of the resistant element is below a predetermined threshold, moisture detection sensor 150, wearing sensor 130, sensor fusion network 160, and microcontroller 110 may be used to indicate that electronic personal wearable device 105 is being worn. Alternatively, moisture detection sensor 150 may be a capacitive moisture sensor where moisture affects the dialectric constant of the capacitive sensor element to vary current flow, voltage, or another electrical characteristic of a capacitor in the capacitive sensor element.

Pulse detection circuit 155 may include a pulse detector implemented with infrared light emitting diodes connected to sensor fusion network 160 and microcontroller 110. Pulse detection circuit 155 may be implemented as a single electronic package and may incorporate, for example, a Texas Instruments AFE4400 package. Pulse detection circuit 155 may detect a pulse, for example, an infrared pulse which may be sensed through the soft palate or other portion of the mouth of the user. The pulse may be received from a smart phone application or another device and may cause pulse detection circuit 155 to transmit location tracking information through transceiver 120, may indicate that electronic personal wearable device 105 has been installed on a person, may initiate transmission of information collected through wearing sensor 130, or initiate any other function of electronic personal wearable device 105.

Sensor fusion network 160 may serve to receive data from any of ambient temperature sensor 135, skin temperature sensor 140, strain gauge 145, moisture detection sensor 150, and pulse detection circuit 155 simultaneously, continuously, serially, or in parallel. Sensor fusion network 160 may serve to increase a confidence interval for assessing, for example, a wear time for electronic personal wearable device 105. For example, when two sensors simultaneously or within a short time frame, detect that electronic personal wearable device 105 is installed on a person, the confidence interval that electronic personal wearable device 105 is actually installed on a person is higher. Use of wear sensor 130 with sensor fusion network 160 will be discussed in greater detail below.

In one embodiment, electronic personal wearable device 105 may include, as part of energy storage 125, or independently, charging circuitry for receiving and storing energy. Charging circuitry associated with energy storage 125 may be implemented as wired or wireless charging circuitry using a tightly-coupled electromagnetic inductive coil, a radiative electromagnetic resonant charging circuit, or an uncoupled RF charging. Further, charging circuitry may also harvest energy using heat from a person's mouth when installed using thermoelectric or thermionic principles, ambient RF energy, or piezoelectric devices. The charging circuitry may be part of or connected to energy storage 125 and may serve to supply energy to energy storage 125 for storage.

FIG. 2 illustrates a system 200 that facilitates communication between electronic personal wearable device 205, a personal electronic device 215, a server computer 225, and one or more other electronic devices such as personal electronic devices 235/245 and computer 255. Personal wearable device 205 may be similar in implementation and discussion to electronic personal wearable device 105 shown and described above with respect to FIG. 1. Electronic personal wearable device 205 may include an electronic module 210 which may include microcontroller 110, indicator 115, transceiver 120, energy storage 125, and wearing sensor 130 with all of wearing sensor 130 components described above in FIG. 1B.

Personal electronic device 215, labeled as “smartphone” in FIG. 2 may be implemented as a smartphone, a tablet, a laptop computer, a cloud server computer, or any other device which is capable of wireless communication and executing a program application. Personal electronic device 215 may include device communication circuitry which facilitates information communication between transceiver 120 and personal electronic device 215. Device communication circuitry in personal electronic device 215 may execute one or more communication protocols including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between personal wearable device 105 and, for example, personal electronic device 215, or server computer 225

Personal electronic device 215 may further include a microcontroller which may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. The microcontroller may execute pre-programmed instructions to perform functionalities described herein.

Personal electronic device 215 may be programmed with an application 220 which is a series of computer instructions which when executed by the microcontroller, cause the microcontroller to perform a series of actions or a method, such as will be discussed below. Application 220 may include an alert module which may transmit a message using any known communication protocol, including an SMS message, a banner notification, or any other type of notification that an alert has been generated due to proximity separation between the electronic personal wearable device 205 and personal electronic device 215, for example.

Personal electronic device 215 may receive information from electronic personal wearable device 205, such as information stored within a memory associated with an onboard microcontroller 110 or information and/or data generated by wearing sensor 130. Personal electronic device 215 may provide data to application 220 and may also provide the data to a computing server 225, which may be a cloud server, for storage on cloud service 230. Cloud service 230 may provide memory storage which may be accessed by, for example, a user of personal electronic device 215, such as a wearer or a parent/guardian of a wearer of electronic personal wearable device 215, a user of personal electronic device 235 (e.g., an orthodontist), a user of personal electronic device 245 (e.g., orthodontic staff), or a user of computer 255 (e.g., an orthodontist, or orthodontic staff). Users of personal electronic devices 235 and/or 245 and computer 255 may have permission to access information stored on server computer 225 and may use app 240, 250, or web browser 260 to access the information generated by electronic personal wearable device 205. This information may help an orthodontist/ophthalmologist/or other professional, for example, determine how much, or in some cases, how little a user has been wearing a retainer, bridge, eyeglasses, sunglasses, or other electronic personal wearable device.

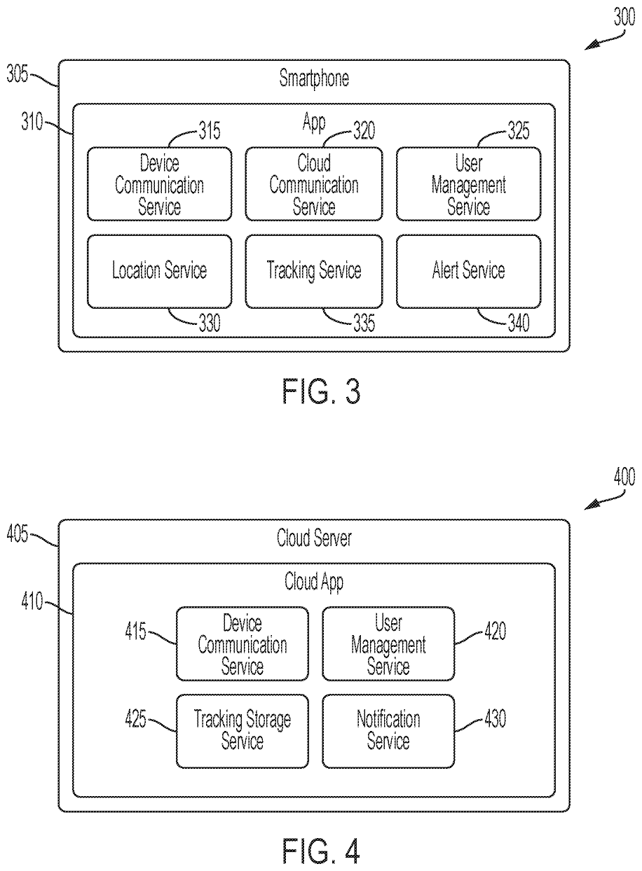

FIG. 3 illustrates a system 300 for communicating with electronic personal wearable device 105. System 300 includes a personal electronic device 305, labeled as “smartphone” in FIG. 3. It is to be noted that personal electronic device 305 may be similar in implementation and description to personal electronic device 205 discussed above with respect to FIG. 2 and may be implemented as a smartphone, a tablet, a laptop computer, a cloud server computer, or any other device which is capable of wireless communication and executing a program application.

Personal electronic device 305 may further include a microcontroller which may include one or more hardware devices which may include hardware components such as a combination of processors, microcontrollers, busses, volatile and non-volatile memory devices, non-transitory computer readable memory devices and media, data processors, control devices, input devices, output devices, network interface devices, and other types of components that are apparent to those skilled in the art. The microcontroller may execute pre-programmed instructions to perform functionalities described herein. Personal electronic device 305 may be programmed with an application 310 which is a series of computer instructions which when executed by the microcontroller, cause the microcontroller to perform a series of actions or a method, such as will be discussed below. The microcontroller may further execute instructions which perform the functions of device communication service 315, cloud communication service 320, user management service 325, location service 330, tracking service 335, and alert service 340, described below.

Application 310 may include a device communication service 315, a cloud communication service 320, a user management service 325, location service 330, tracking service 335, and alert service 340. Communication service 315 may be implemented by device communication circuitry which facilitates information communication between transceiver 120 and personal electronic device 215/305. Cloud communication service 320 may be implemented by the same device communication circuitry which facilitates information communication between personal electronic device 215/305 and server computer 225. Device communication circuitry which implements device communication service 315 may execute one or more communication protocols including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between electronic personal wearable device 105 and, for example, personal electronic device 215/305 by device communication service 315, or server computer 225 by cloud communication service 320. Device communication service 315 may maintain regular communication with electronic personal wearable device 105, cause an alert to be generated if connection is lost with the device, and notify the wearer that electronic personal wearable device 105 is not present. Cloud communication service 320 may manage communication to the cloud between personal electronic device 215/305 and computer server 225. As personal electronic device 215/305 receives and loses connection to a communication protocol (e.g., LTE/4G/WiFi networks), cloud communication service 320 may queue transmission of information and data from both electronic personal wearable device 105 and personal electronic device 215/305, such as location information, to a server computer. Personal electronic device 215 may, via cloud communication service 320 also receive information from server computer 225, including location information, for a last known location of electronic personal wearable device 105.

Personal electronic device 305 may further include a user management service 325 within application 310. User management service 325 may allow a user of personal electronic device 305 selective access to information and data generated by electronic personal wearable device 105 and 205, shown in FIG. 1 or 2. One example of selective access may be a password setting, a passcode setting, biometric identification, or other authentication or identification parameters. User management service 325 may further distinguish an identifier associated with one of electronic personal wearable device 105/205 and another one of electronic personal wearable device 105/205 to distinguish one wearer of electronic personal wearable device 105/205. For example, a parent who obtains information from electronic personal wearable device 105/205 via personal electronic device 305 may have two children who both wear orthodontic retainers. User management service 325 may indicate to a parent which information and data was derived from which child's electronic personal wearable device 105/205. User management service 325 may further allow a user of personal electronic device 305 to set permissions for which other devices, such as personal electronic devices 235/245 and computer 255 may access or be notified of information and data generated by electronic personal wearable device 105/205.

Personal electronic device 305 may further provide a location service 330 via application 310. Location service 330 me be a portion of application 310 which tracks the physical location of both the wearer of electronic personal wearable device 105/205 and the location of electronic personal wearable device 105/205. Location service 330 may poll electronic personal wearable device 105/205 on demand or at a predetermined interval to detect whether or not electronic personal wearable device 105/205 is within communication range. Location service 330 may log the physical location of electronic personal wearable device 105/205 in response to each poll request and a time associated with the request.

Personal electronic device 305 may further provide a tracking service 335 via application 310. Tracking service 335 may be a portion of application 310 which tracks the physical location of electronic personal wearable device 105/205 based on information from wearing sensor 130 and sensors within personal electronic device 305. For example, personal electronic device 305 may monitor a location context and map regular locations for the wearer such as “English Class,” “Lunchroom,” “Home,” and etc. Personal electronic device 305 may monitor a compass bearing and footsteps as a basis for judging a distance and bearing from a certain location. Personal electronic device 305 may include an accelerometer to detect footsteps and a bearing sensor to detect a magnetic bearing from a certain location. Personal electronic device 305 may further use machine learning to identify locations frequented by a user, such as “Home,” and adjust thresholds for alerts triggered by alert service 340 (discussed below) based on the specific location. For example, when at home, the wearer may remove an orthodontic retainer and keep the orthodontic retainer in a container in the user's bedroom during a dinner meal. Based on machine learning, personal electronic device 305 may adjust an alarm threshold to identify that electronic personal wearable device 105/205 is farther away from the user than normal although because the wearer is at home, it is unlikely that electronic personal wearable device 105/205 is lost. Further, an electronic personal wearable device 105/205 may be placed within a cleaning and charging device where cleaning and charging of electronic personal wearable device 105/205 is taking place. Tracking service 335 may identify that personal wearable device 105/205 is in a charging or cleaning state and fail to initiate an alert by alert service 340.

Personal electronic device 305 may further provide an alert service 340 as part of application 310. Alert service 340 may identify through location service 330 and tracking service 335 that electronic personal wearable device 105/205 has left the proximity of personal electronic device 305 (i.e., because personal electronic device 305 has lost communication with electronic personal wearable device 105/205 or a predetermined threshold distance between personal electronic device 305 and electronic personal wearable device 105/205 has been exceeded). Personal electronic device 305 may also receive information generated by wear sensor 130 that the wearer and the personal electronic device 305 are not in motion with each other, electronic personal wearable device 105/205 is not installed within the wearer's mouth, or that the temperature exceeds a certain threshold. Alert service 340 may, in this case, cause an alert to be provided by the personal electronic device 305 that alerts the user of personal electronic device 305 that electronic personal wearable device 105/205 has left the proximity of the person at the time a communication link between them is lost. Alert service 340 may, in some cases, cause cloud communication service 320 to transmit an alert to cloud server 255 that identifies electronic personal wearable device 105/205 has been lost. Alert service 340 may initiate an alert manually if electronic personal wearable device 105/205 is within range (e.g., lost but still within communication range of personal electronic device 305).

FIG. 4 illustrates a system 400 for storing information generated by electronic personal wearable device 105/205. System 400 includes a server computer 405, labeled as “cloud service” in FIG. 4. Server computer 405 may be implemented as a server device associated with a cloud computing system and may be similar in implementation and description to server computer 225, shown in FIG. 2 and described above.

Server computer 405 may further include a microprocessor which may include one or more hardware devices which may include hardware components such as a combination of processors, microprocessor, busses, volatile and non-volatile memory devices, non-transitory computer readable memory devices and media, data processors, control devices, input devices, output devices, network interface devices, and other types of components that are apparent to those skilled in the art. The microprocessor may execute pre-programmed instructions to perform functionalities described herein. Server computer 405 may be programmed with an application 410 which is a series of computer instructions which when executed by the microprocessor, cause the microprocessor to perform a series of actions or a method, such as will be discussed below. The microprocessor may further execute instructions which perform the functions of device communication service 415, user management service 420, tracking storage service 425, and notification service 430, described below.

Device communication service 415 may be implemented by device communication circuitry which facilitates information communication between server computer 405 and one or more of personal electronic device 215/305. Device communication circuitry which implements device communication service 415 may execute one or more communication protocols including NFC (Near Field Communication), RFID (RF ID tag), Wi-Fi, BLE (Bluetooth Low Energy), ZigBee, Z-Wave, RF (Radio Frequency), RF4CE, Ethernet, telephone line, cellular channels, or others that operate in accordance with protocols defined in IEEE (Institute of Electrical and Electronics Engineers) 802.11, 801.11a, 801.11b, 801.11e, 802.11g, 802.11h, 802.11i, 802.11n, 802.16, 802.16d, 802.16e, or 802.16m using any network type including a wide-area network (“WAN”), a local-area network (“LAN”), a 2G network, a 3G network, a 4G network, a 5G network, a Worldwide Interoperability for Microwave Access (WiMAX) network, a Long Term Evolution (LTE) network, Code-Division Multiple Access (CDMA) network, Wideband CDMA (WCDMA) network, any type of satellite or cellular network, or any other appropriate protocol to facilitate communication between personal electronic device 215/305 and server computer 405. Device communication service 415 may maintain regular communication with personal electronic device 215/305, cause notification to be generated if connection is lost with the device, and notify the wearer and other/all users identified to receive notifications that electronic personal wearable device 105 is lost. Device communication service 415 may manage communication between the server computer 225/405 and personal electronic device 215/305 and computer server 225. Personal electronic device 215/305 may, via device communication service 415 also receive information from server computer 225, including location information, for a last known location of electronic personal wearable device 105.

Server computer 405 may further include a user management service 420 within application 410. User management service 420 may allow a user of server computer 225/405 selective access to information and data stored within server computer 405 in a cloud computing system. One example of selective access may be a password setting, a passcode setting, biometric identification, or other authentication or identification parameters. User management service 420 may further distinguish an identifier associated with one of electronic personal wearable device 105/205 and another one of electronic personal wearable device 105/205 to distinguish one wearer of electronic personal wearable device 105/205. For example, a parent who obtains information from server computer 225/405 via personal electronic device 215/305/235/245 may have two children who both wear orthodontic retainers. User management service 420 may indicate to a parent which information and data was derived from which child's electronic personal wearable device 105/205. User management service 420 may further allow a user of server computer 405 to set permissions for which other devices, such as personal electronic devices 235/245 and computer 255 may access or be notified of information and data generated by electronic personal wearable device 105/205.

Server computer 405 may further provide a tracking storage service 425 via application 410. Tracking storage service 425 may be a portion of application 410 which stores information related to the physical location of electronic personal wearable device 105/205 based on information from wearing sensor 130 and sensors within personal electronic device 215/305 which is received from personal electronic device 215/305. For example, data accumulated by tracking service 335, discussed above with respect to FIG. 3, may be transmitted through cloud communication service 320 in personal electronic device 305 to tracking storage service 425 in cloud server 405 for information storage.

Server computer 405 may further provide a notification service 430 as part of application 410. Notification service 430 may receive an alert from alert service 340 of personal electronic device 305 that an electronic personal wearable device has been moved outside the proximity of personal electronic device 305. In response, notification service 430 may generate a notification that is sent to any personal electronic device associated with the user, such as those identified for receiving a notification through user management service 420. For example, the wearer's parents, orthodontist, orthodontic staff, and other identified individuals may receive a notification via personal electronic devices 240/250 and computer 255, for example.

In use, a user may wear electronic personal wearable device 105, such as a retainer. The user may be required to wear the retainer at all times with the exception of eating. Since many people who wear retainers may be relatively young and not appreciate the monetary cost of making a retainer, such users may be less careful about where the retainer is placed. During school lunch, a user may remove a retainer to eat a meal and set the retainer on a lunch tray to eat lunch. The hypothetical youthful user of the retainer may attempt to rush through lunch to join friends outside for a recess period at school and inadvertently place the retainer in the trash with the leftovers of the lunch without realizing the retainer has been placed in the trash.

For purposes of this discussion, the user may be notified by personal electronic device 215/305, that the retainer has left the proximity of the personal electronic device 215/305 (e.g. the user's person). Functionally, personal electronic device 215/305 may, via application 310 executed by a microcontroller, detect that personal electronic device 215/305 is no longer receiving, for example, an NFC signal from RF transceiver 120 of electronic personal wearable device 105 and cause, as a result, an alert to be provided to the user via alert service 340 in personal electronic device 215/305 that electronic personal wearable device 105 has been misplaced, at the time the user has misplaced electronic personal wearable device 105. Should the user not notice the alert that electronic personal wearable device 105 has been misplaced, application 310 on personal electronic device 215/305 may provide the user with an indication of a last known location of electronic personal wearable device 105 via location service 330 and tracking service 335 which may identify where the retainer was lost and provide left, right, and distance directions to recover electronic personal wearable device 105. When personal electronic device 215/305 reconnects with electronic personal wearable device 105 via transceiver 120, indicator 115 may be actuated to provide an indicator of the precise location of electronic personal wearable device 105 to allow electronic personal wearable device 105 to be retrieved.

In the event that the young user has misplaced a personal electronic device 215/305, location and tracking information may be retrieved from cloud server 405 and tracking storage service 415 via device communication services 415 and cloud communication service 320 to use another one of personal electronic device 235/245 and/or computer 255 to locate electronic personal wearable device 105. If electronic personal wearable device 105 has not been located within a specific amount of time or is otherwise unrecoverable, notification service 430 of server computer 405 may send a notification to personal electronic device 235/245 and/or computer 255 which indicates that electronic personal wearable device 105 has been lost. Specific techniques for tracking and locating electronic personal wearable device 105 are discussed below.

A further useful advantage of the disclosed device and system is that electronic personal wearable device 105 and personal electronic device 215/305 may perform wear time tracking. Some electronic personal wearable devices may be worn for years or a lifetime. In the case of an orthodontic retainer, for example, if the retainer is not worn for a sufficient amount of time on a frequent basis, teeth in the wearer's mouth may begin to move out of place. After teeth have moved out of place, the retainer may become more and more uncomfortable to wear which results in orthodontic patients to stop using their retainer and lose the benefit of years of orthodontic braces. Thus, in order to incentivize orthodontic patients to wear a retainer, for example, personal electronic device 215/305 may recommend an optimal wear time for a wearer of electronic personal wearable device 105 to wear electronic personal wearable device 105. Recommendations may be suggestions based on current wear time information detected from electronic personal wearable device 105 and may be the result of machine learning to identify or suggest to a user that additional wear time is necessary, include rankings of wear time as compared with other wearers of an electronic personal wearable device, and/or provide real-time suggestions for installing and using an electronic personal wearable device. Personal electronic device 215 may perform machine learning or receive information from server computer 405 which is based on machine learning and cloud based analytics to identify an optimal wear time for a specific user (which may be different from another user), based on feedback or information initially generated by wearing sensor 130. For example, strain gauge 145 may detect an amount of tension or pressure exerted on electronic personal wearable device 105 when moisture detection sensor 150 indicates that electronic personal wearable device 105 is in a wearer's mouth. Data from strain gauge 145 and moisture detection sensor 150 may be collected by sensor fusion network 160 and transmitted by microcontroller 110 through transceiver 120 to personal electronic device 215/305 and from personal electronic device 215/305 to server computer 405. Data provided to server computer 405 may be analyzed to determine an amount of time a wearer should wear electronic personal wearable device 105 to produce a specified result. For example, when electronic personal wearable device 105 is an orthodontic retainer, computer server 405 may analyze strain information on the retainer and actual wear time and calculate an optimal amount of time the wearer should wear the retainer to reduce the strain on the retainer.

Other benefits of this disclosure are that the wearer's parents, orthodontist, orthodontic staff, and other people who have permission to view data from electronic personal wearable device 105 may identify when a retainer is not being worn frequently enough, for example. In many cases, when teeth have moved to the point where a retainer causes discomfort, wearing sensor 130 information can be used by computer server 405 to measure for a series of temporary retainers to move teeth back into the ideal anatomical position for an original retainer. Any information obtained from wearing sensor 130 may be used to assess actual wear time, determine optimal wear time for a particular user, and suggest behavior modifications to enhance the effectiveness of electronic personal wearable device 105.

Server computer 405 may be connected to a cloud service, as previously discussed which may provide analysis tools for compiling data from wearing sensor 130 of electronic personal wearable device 105. The analysis tools may perform statistical regressions on data for that user, for a plurality of users, or for all users of electronic personal wearable device 105. Data gathered across a plurality of users may be useful in identifying average wear times as compared to results of wearing electronic personal wearable device 105 and provide other statistical or other information for improving the wearing experience for a wearer of electronic personal wearable device 105. Various methods disclosed below identify exemplary implementations of the foregoing functionality.

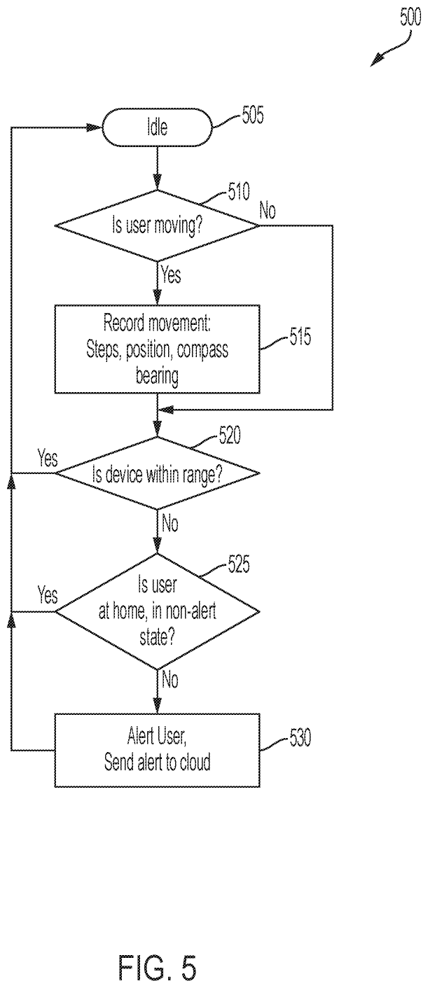

FIG. 5 illustrates a method 500 for tracking a location of electronic personal wearable device 105 by personal electronic device 215/305. Method 500 begins at step 505 where personal electronic device 215/305 is idle. Personal electronic device 215/305 may or may not, by a microcontroller, detect user movement at step 510. If movement by a user has been detected (step 510—“Yes”), personal electronic device 215/305 may record the movement, number of steps, position, and compass bearing. If no movement by a user has been detected (step 510—“No”), method 500 proceeds to step 520.

At step 520, personal electronic device 215/305 may ping or poll electronic personal wearable device 105 to determine whether electronic personal wearable device 105 is within range of personal electronic device 215/305. If a response is provided by electronic personal wearable device 105, at step 520 (step 520—“Yes”), personal electronic device 215/305 may return to step 505 and remain idle until movement is detected again. If a response is not provided by electronic personal wearable device 105 at step 520 (step 520—“No”), personal electronic device 215/305 may determine whether or not the user/personal electronic device 215/305 are in a known location, such as at home at step 525. If personal electronic device 215/305 determines at step 525 that electronic personal wearable device 105 and personal electronic device 215/305 are in a known location and not in an alert state (step 525—“Yes”), personal electronic device 215/305 may return to an idle state until movement is detected again. If at step 525, personal electronic device 215/305 is not in a known location (step 525—“No”), personal electronic device 215/305 may generate an alert by alert service 340 and transmit the alert to cloud server 405 which may then provide a notification to other personal electronic devices, such as personal electronic devices 235/245 and/or computer 245. In this example, personal electronic device 215/305 may constantly monitor a location of electronic personal wearable device 105 and alert a wearer when electronic personal wearable device 105 is not within communicable range of personal electronic device 215/305.

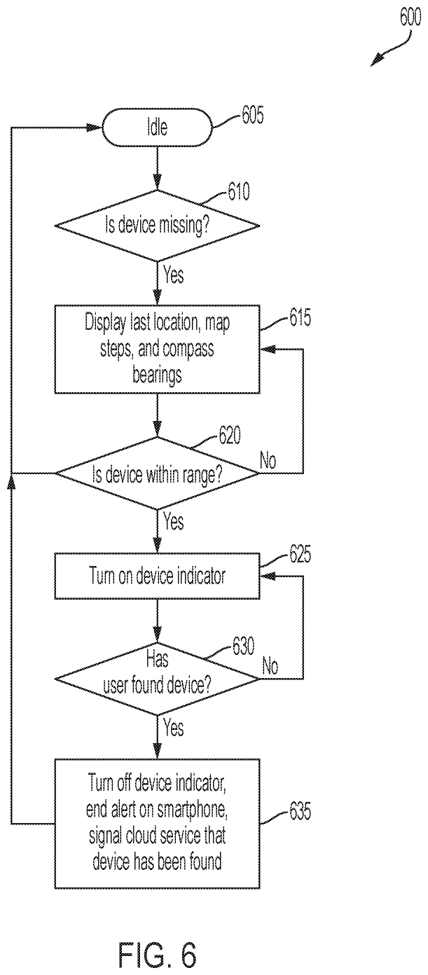

FIG. 6 illustrates a method 600 for tracking a location of personal wearable device 105 by personal electronic device 215/305. Method 600 begins at step 605 where personal electronic device 215/305 is idle. Personal electronic device 215/305 may, by a microcontroller, detect that electronic personal wearable device 105 is missing at step 610. In response to detecting that electronic personal wearable device 105 is missing at step 610, personal electronic device 215/305 may cause, at step 615, a display to be provided that visually indicates a last known location, a map, number of steps from the last known location of electronic personal wearable device 105, and compass bearings to the last known location of electronic personal wearable device 105. At step 620, personal electronic device 215/305 may attempt to communicate with electronic personal wearable device 105 to determine if electronic personal wearable device 105 is within communication range. If electronic personal wearable device 105 is not within communication range (step 620—“No”), personal electronic device 215/305 may continue to display a last known location, a map, number of steps from the last known location of electronic personal wearable device 105, and compass bearings to the last known location of electronic personal wearable device 105.

If electronic personal wearable device 105 is within communication range of personal electronic device 215/305 (step 620—“Yes”), personal electronic device 215/305 may instruct electronic personal wearable device 105 to turn on indicator 115 at step 625. As previously discussed, indicator 115 may be a light emitting diode, a piezoelectric speaker, or other indicator, and help a user locate electronic personal wearable device 105. At step 630, personal electronic device 215/305 queries the user to determine whether or not electronic personal wearable device 105 has been found. If electronic personal wearable device 105 has not been found (step 630—“No”), personal electronic device 215/305 may again instruct electronic personal wearable device 105 to turn on indicator 115 or, alternatively, increase a relative brightness of a light emitting diode or a volume of a piezoelectric speaker, or otherwise increase an intensity of indicator 115. If the user indicates to personal electronic device 215/305 that electronic personal wearable device 105 has been found (step 630—“Yes”), personal electronic device 215/305 may instruct electronic personal wearable device 105 to turn off indicator 115, discontinue an alert on personal electronic device 215/305, and send a message to cloud server 405 that electronic personal wearable device 105 has been found at step 635.

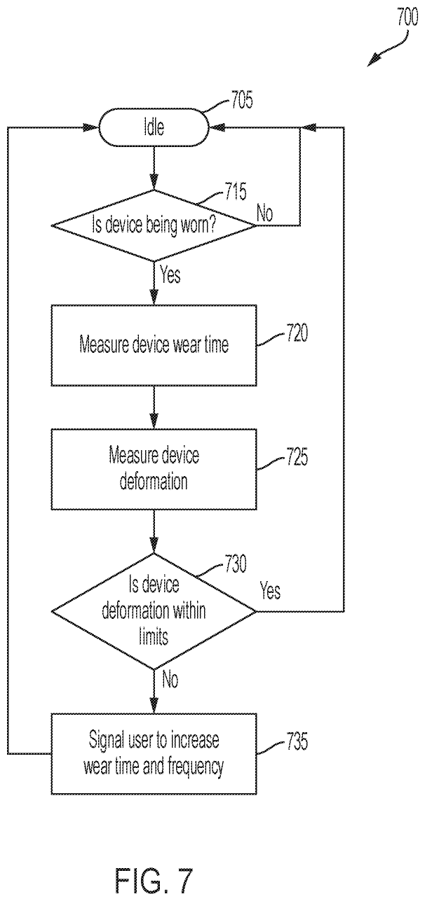

FIG. 7 illustrates a method 700 for tracking wear time of electronic personal wearable device 105 by personal electronic device 215/305. Method 700 begins with personal electronic device 215/305 in an idle state which periodically queries electronic personal wearable device 105 for communications information and for data generated by wearing sensor 130. At step 715, personal electronic device 215/305 queries electronic personal wearable device 105 to receive data from wearing sensor 130. Wearing sensor 130 may provide data generated by one or more sensors in personal wearable device 105 by microcontroller 110 and transceiver 120. The data generated bythe one or more sensors may be provided to personal electronic device 215/305 which analyzes the data to determine whether or not electronic personal wearable device 105 is being worn at step 715. If electronic personal wearable device 105 is not being worn, based on analyzed data generated by wearing sensor 130, (step 715—“No”), method 700 returns to an idle state. However, if electronic personal wearable device 105 determines, based on analyzed data generated by wearing sensor 130, that electronic personal wearable device 105 is being worn (step 715—“Yes”), personal electronic device 215/305 may begin to measure device wear time at step 720. At step 725, personal electronic device 205/315 may receive additional data generated by wearing sensor 130 to measure deformation of electronic personal wearable device 105. At step 730, personal electronic device 205/315 may analyze the data generated by wearing sensor 130 to determine whether or not electronic personal wearable device 105 is within deformation limits. The deformation limits may be associated with the movement of teeth, for example, during use of an orthodontic retainer. As teeth move, electronic personal wearable device 105 may experience deformation from strain exerted on electronic personal wearable device 105 by a person's teeth. If electronic personal wearable device 105 is within deformation limits, (step 730—“Yes”), personal electronic device 205/315 may return to an idle state at step 705. If electronic personal wearable device 105 is not within deformation limits (step 730—“No”) personal electronic device 205/315 may signal the wearer to increase wear time and frequency. In this example, an optimal wear time may be provided to the wearer of electronic personal wearable device 105. The signal to the wearer via personal electronic device 205/315 may suggest that the wearer wear electronic personal wearable device 105 for an additional amount of time or that the user has not worn the device in a period that is longer than suggested. Machine learning techniques may also be applied in analyzing whether or not electronic personal wearable device 105 is within deformation limits. Wear time and deformation data may be stored in server computer 405, aggregated, and analyzed to improve the machine learning model.

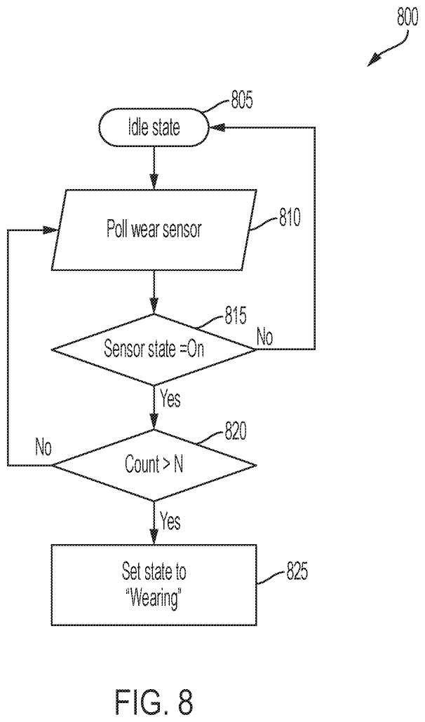

FIG. 8 illustrates a method 800 for identifying that electronic personal wearable device 105 is being worn. At step 805, personal electronic device 215/305 may be in an idle state. At step 810, personal electronic device 215/305 may poll wearing sensor 130 within electronic personal wearable device 105 to determine whether or not electronic personal wearable device 105 is being worn at step 815. If personal electronic device 215/305 determines that data from wearing sensor 130 indicates that electronic personal wearable device 105 is not being worn (step 815—“No”), personal electronic device 215/305 returns to an idle state at step 805. If, however, personal electronic device 215/305 determines that data from wearing sensor 130 indicates that electronic personal wearable device 105 is being worn (step 815—“Yes”), method 800 moves to step 820 to determine whether a count of the times step 815—“Yes” has been performed exceeds a threshold number “N”. If the count exceeds threshold number “N” at step 820 (step 820—“Yes”), the state of electronic personal wearable device 105 is set to wearing at step 825 and receives periodic information from wearing sensor 130. If the count does not exceed a threshold number “N” at step 820 (step 820—“No”), method 800 returns to step 810.

The foregoing description has been presented for purposes of illustration. It is not exhaustive and does not limit the invention to the precise forms or embodiments disclosed. Modifications and adaptations will be apparent to those skilled in the art from consideration of the specification and practice of the disclosed embodiments. For example, components described herein may be removed and other components added without departing from the scope or spirit of the embodiments disclosed herein or the appended claims.

Other embodiments will be apparent to those skilled in the art from consideration of the specification and practice of the disclosure disclosed herein. It is intended that the specification and examples be considered as exemplary only, with a true scope and spirit of the invention being indicated by the following claims.

What is claimed is:

1. A device, comprising:
a processor,
a transceiver, and
a wearing sensor which indicates the device is being worn by a user when the device is worn by the user.

2. The device of claim 1, wherein the wearing sensor includes an ambient temperature sensor.

3. The device of claim 1, wherein the wearing sensor includes a skin temperature sensor.

4. The device of claim 1, wherein the wearing sensor includes a strain gauge.

5. The device of claim 1, wherein the wearing sensor includes a moisture detection sensor.

6. The device of claim 1, wherein the wearing sensor includes a pulse detection circuit.

7. The device of claim 1, wherein the wearing sensor includes a sensor fusion network to receive data from one or more sensors within the wearing sensor.

8. The device of claim 1, further comprising:
an indicator.

9. The device of claim 1, further comprising:
an energy storage device which includes a wireless charging circuit.

10. A system comprising:
an electronic personal wearable device, comprising.
a processor,
a transceiver, and
a wearing sensor which indicates the device is being worn by a user when the device is worn by the user; and
a personal electronic device.

11. The system of claim 10, wherein data generated by the wearing sensor is transmitted to the personal electronic device.

12. The system of claim 11, wherein data generated by the wearing sensor includes wear time data.

13. The system of claim 11, wherein data generated by the wearing sensor includes location data.

14. The system of claim 13, wherein, in response to receiving location data, the personal electronic device generates tracking information for the electronic personal wearable device.

15. The system of claim 10, wherein the personal electronic device transmits a message to a user in response to losing a communication link to the electronic personal wearable device when the personal electronic device determines that the communication link has been lost.

16. The system of claim 15, wherein the personal electronic device provides a compass bearing to a last known location of the electronic personal wearable device.

17. The device of claim 16, wherein the personal electronic device indicates a number of steps between the electronic personal wearable device and the personal electronic device.

18. The system of claim 17, wherein the personal electronic device restores a communication link to the electronic personal wearable device.

19. The system of claim 18, wherein the personal electronic device instructs the electronic personal wearable device to turn on an indicator.

20. A device, comprising:
an orthodontic retainer, the orthodontic retainer comprising:
a processor,
a transceiver, and
a wearing sensor which indicates the orthodontic retainer is being worn by a user when the orthodontic retainer is worn by the user.
